(19) United States
(12) Patent Application Publication
(10) Pub. No.: US 2007/0009079 A1
(43) Pub. Date: Jan. 11, 2007

(54) Method for automatically setting and reconstructing the field of view along the body boundaries on a CT topogram

(76) Inventors: Ma Hao Chen, Shanghai (CN); Xiao Dong Xu, Shanghai (CN); Feng Zhao, Shanghai (CN)

Correspondence Address: HARNESS, DICKEY & PIERCE, P.L.C., P.O.BOX 8910, RESTON, VA, 20195, US

(21) Appl. No.: 11/476,838

(22) Filed: Jun. 29, 2006

(30) Foreign Application Priority Data
Jun. 30, 2005 (CN) 200510080218.3

(51) Int. Cl. H05G 1/60; A61B 6/00; G01N 23/00; G21K 1/12

(52) U.S. Cl. 378/4

(57) ABSTRACT

A method is disclosed for automatically setting and reconstructing the field of view along the body boundaries on a CT topogram, the constructed field of view including exactly all the points on the body boundaries inside the scan area on the topogram. In at least one embodiment of the method, the search takes place from the points of the body bones in the vicinity of the body boundaries, in the direction outward, and the body boundaries of the patient are determined by comparing the CT values. At least one embodiment of the method can be used for a precise, highly efficient and quick setting and reconstruction of the field of view.

### PRIORITY STATEMENT

The present application hereby claims priority under 35 U.S.C. §119 on Chinese patent application number 200510080218.3, filed Jun. 30, 2005, the entire contents of which is hereby incorporated herein by reference.

### FIELD

The present invention generally relates to a method for setting and reconstructing the field of view (termed “FOV” below for brevity) on the topogram (also termed “Topo image”) for computer tomography (termed “CT” below for brevity). For example, it may relate to a method for automatically setting the FOV along the left-hand and right-hand body boundaries (contours) of a patient on a CT topogram.

### BACKGROUND

Before beginning to carry out the serial scanning or the spiral scanning, there is normally a need with CT units to create a positioning image for the patient in order to determine the scan area with the aid of this positioning image, and in order to be able to undertake the positioning of the image reconstruction. Subsequently, an x-radiation source is used to perform the scanning operation of the area of the patient's body to be examined so as to carry out the image reconstruction and to produce the medical image with the aid of the projection data.

During a topogram scan, the spherical tube, which serves as x-radiation source inside the CT unit, remains with its position unchanged. See FIG. 1 in this regard. In the relevant figure, the part designated by the number 110 is the area scanned in a typical topogram 100. The part designated by number 112 is the unscanned area in the topogram.

In the current known systems, the area to be scanned and the FOV are set on the topogram 100 designated above after the latter has been obtained, and this is illustrated by a rectangle 20. Under particular conditions, a parallelogram can also be used for the illustration.

In this case, the scan area is determined along the side of the height of the patient's body 60 (that is to say the vertical side of a rectangle). The relevant examination area in the scan area and in the reconstructed image is acquired by using the scan area designated above, while the other areas of a patient's body are not scanned.

By contrast, the FOV is determined along the side of the width direction of a patient's body (that is to say the horizontal side of the rectangle). The FOV designated above is used to determine the display area of the image. Thus, the determination of position and size of the display of the area being examined on the image. The CT unit subsequently performs this serial scanning or the spiral scanning as well as the image reconstruction in accordance with the area designated above and set in a rectangular shape.

It is known from the findings of clinical practice that in numerous CT examinations the FOV is normally set along the boundaries 62 of the patient's body 60 in order to be able to obtain an optimum display area and a corresponding result. In the case of the technique currently to hand, the scan area and the FOV are set altogether in advance to a specific position. The corresponding size is also fixed in advance, as the rectangle 20 from FIG. 1 shows. However, the width of the patient's body that is being scanned differs in each case.

Consequently, the operating staff of the CT unit must firstly fix the size of the scan area on the topogram of the relevant patient (that is to say the distance between the vertical sides of the rectangle 20), in order subsequently to set the size of the setting range for the FOV (that is to say the distance between the horizontal sides of the rectangle 20) in accordance with the actual conditions regarding the width of the patient's body. It is achieved thereby that the area of the FOV includes exactly all the body boundaries inside the scan area designated above.

The setting designated above for the FOV by manual regulation entails a high degree of deficiency. Moreover, in order to obtain an optimum display area and an appropriate result there is firstly a need to ensure that the boundaries of the FOV intersect the points of the outermost sides of the left-hand and right-hand body boundaries of the patient. The boundaries of the manually set FOV are in no way adequately precise. Moreover, the manual setting of the boundaries of the FOV requires a high outlay on time, and this leads to a corresponding lengthening of the period required for the overall operation, and reduces the affectivity. Finally, the lengthening, designated above, of the overall operation leads to an additional physiological and psychological burden on the patient.

### SUMMARY

In at least one embodiment of the present invention, a method is provided for automatically setting the FOV along the body boundaries on a topogram which enables a precise, highly efficient and quick setting of the FOV.

A method for automatically setting and reconstructing the field of view along the body boundaries is proposed according to at least one embodiment of the invention in order to carry out setting and reconstruction of the FOV inside a specific scan area of the topogram. The method, in at least one embodiment, includes:
a) searching for the maximum CT value of each scan line inside the scan area on the topogram designated above;
b) searching along the relevant scan line from outside the body in the direction inward for points whose CT value is the X1-fold of the maximum CT value designated above, 0<X1<0.5 and these points being located on the body bones in the vicinity of the body boundaries;
c) searching from the points, described in step b), on the body bones in the vicinity of the body boundaries in the direction outside the body for the minimum CT value of the relevant scan line on the topogram;
d) searching from the points, described in step b), on the body bones in the vicinity of the body boundaries in the direction outside the body for points whose CT value is the X2-fold of the minimum CT value designated above, 0.5<X2<1 and these points of the relevant scan line being located on the body boundaries;
e) repeating steps a) to d) to search for points on the body boundary inside the scan area designated above; and
f) setting and reconstructing the field of view such that the field of view includes all found points on the body boundaries inside the relevant scan area.

A very good recognition effect is obtained here when the value for X1 is selected as 0.25 under step b). A very good recognition effect is obtained when the value for X2 is selected as 0.75 under step d). The points with a minimum CT value as described in step c) are located on the outsides of the body boundaries.

When the setting and reconstruction of the field of view is carried under step f), it is ensured that the boundaries of the reconstructed field of view intersect the points of the outermost side on the body boundaries inside the scan area. It is ensured in this way that the reconstructed field of view includes all the points on the body boundaries inside the relevant scan area.

When the maximum CT value described under step a) is greater than a predetermined metal CT value, it is assumed that metal is located in the relevant scan line, and in this case the boundary points of the previous scan line are considered as boundary points of the present scan line.

In a practical implementation example in accordance with at least one embodiment of the present invention, boundary points of the last scan line of an already scanned area are lengthened and form the boundary of an area not yet scanned. It is also possible that the data of a few lines of the start region and end region of the scan area of the topogram are erased in order to prevent deformation of linear artifacts.

In a further practical implementation example of at least one embodiment of the present invention, median filtering is undertaken in order in the course of the search operation to remove found discrete points that correspond to the relevant conditions.

After the body boundaries of the patient have been found in accordance with at least one embodiment of the present invention, it is possible to carry out the precise, highly efficient and quick setting of the FOV on the relevant topogram.

### BRIEF DESCRIPTION OF THE DRAWINGS

FIG. 1 is an overview of the manual setting of the FOV on a CT topogram in accordance with the present prior art.

FIG. 2 is an overview of the automatic setting of the FOV along the body boundaries on a CT topogram in accordance with at least one embodiment of the present invention.

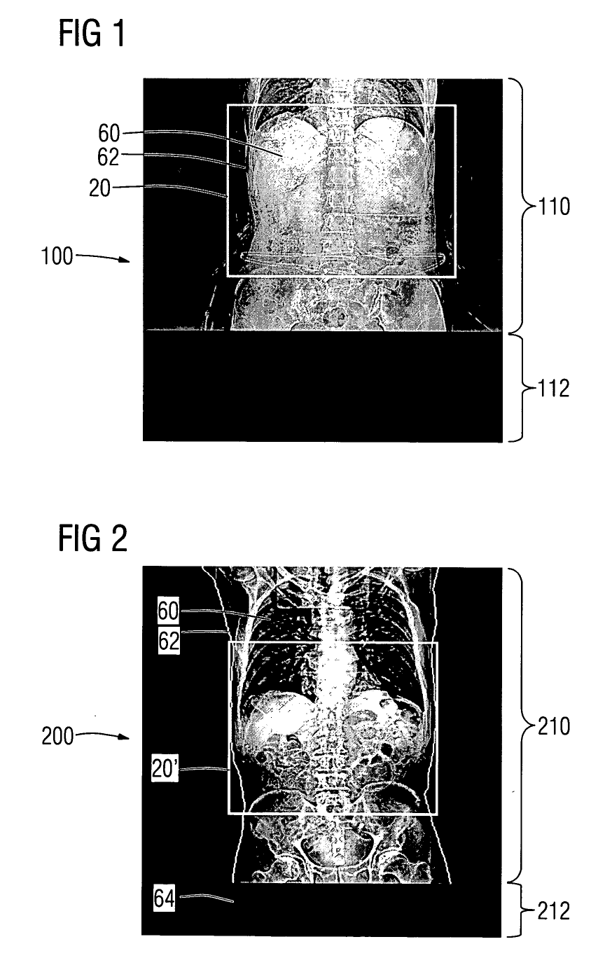

### DETAILED DESCRIPTION OF THE EXAMPLE EMBODIMENTS

The images of the topogram taken by CT units are displayed by way of various brightness levels. The various absorptivities of the body organs and of the body tissue with regard to x-rays are reproduced in this way. Dark shaded regions signify a low absorption on the topogram. These are regions of low density, for example lungs. Bright regions, by contrast, signify a high absorption. These are regions of high density, for example bones. In practice, it is normal to use CT values in order to display the intensity of the relevant density. CT values are normally given in a rising fashion on the topogram for air, fat, water, soft tissue and bone.

The first step in at least one embodiment the present invention is to set the relevant scan area on the topogram. Subsequently, the deviation specifications of the CT values of various regions of the patient's body are used for the purpose of determining the body boundaries of the relevant patient, and then to undertake the setting of the FOV to the body boundaries of the patient inside the scan area such that there is an overlap with the points on the outermost side of the left-hand and right-hand body boundaries inside the scan area. An optimum result can be achieved in this way with regard to scanning and image reconstruction. For this reason, the main task of the automatic setting of the FOV resides in being able to undertake a precise localization of the left-hand and right-hand body boundaries of the patient on the topogram.

In at least one embodiment the present invention, body boundaries include the outer contour lines on the left-hand and the right-hand body sides of the patient on the topogram. The body boundaries inside the scan area are the outer contour lines on the left-hand and the right-hand body sides of the patient inside the scan area set on the topogram previously designated.

In at least one embodiment the present invention, the points of the body boundaries of the patient on the left-hand and right-hand sides are determined along the body height of the patient. Here, the examination area is the region of the scan area set on the topogram designated above. The rate of linear artifacts in the regions where scanning starts and ends is normally higher than in the middle regions. The reason that this is that the boundary lines of the human body normally have no sudden changes and so the erasure of the data of a few scan lines from start and end regions does not entail any negative effects on the precision of the body boundaries in accordance with at least one embodiment the present invention.

Since, in addition, the body of the relevant patient is chiefly located in the middle part of the topogram, in the case of the present invention every scan line of the image is simultaneously subdivided into a right-hand and a left-hand part. The points on the left-hand body boundary of the patient, and the points on the right-hand body boundary of the patient can respectively be found on the left-hand part, designated above, of a scan line and, respectively, on the right-hand part of a scan line. Because the method for searching for the left-hand body boundary of a patient is identical to that for searching for the right-hand body boundary of the patient, only the description of the inventive method for finding the left-hand body boundary is set forth below as an example design.

FIG. 2 serves for reference. In the relevant figure, number 210 signifies the scanned region on the topogram 200. Number 212, by contrast, signifies the unscanned region on the topogram 200. Finding the left-hand body boundaries of the patient by way of the method according to an embodiment the invention includes:
a) searching for the maximum CT value of each scan area inside the scan area on the topogram 200 designated above. The bones of the human body normally offer the highest CT values.
b) searching along the relevant scan line from left to right—that is to say from outside the body 60 of the patient on the topogram 200 designated above in the direction inward for points whose CT value is the X1-fold of the maximum CT value designated above, 0<X1<0.5. The CT values of various parts and tissues of the human body certainly differ, but there is a proportional relationship nevertheless. It is known from numerous experiments that points whose CT value is from 0-0.5 times the maximum CT value are located on the bones of the body 60 in the vicinity of the body boundaries. An optimum recognition effect is achieved in practical application when the value of 0.25 is selected for X1. The reason why the search is to be carried out from outside the body 60 inward is that it is possible in this way to find the points that are located on the bones of the body 60 in the vicinity of the body boundaries, and that the points which are located on the bones of the body 60 in the vicinity of the interior of the body are not found.
c) Searching from the points, described in step b), on the bones of the body 60 in the vicinity of the body boundaries to the left—that is to say in the direction outside the body 60—for the minimum CT value of the part designated above. The found points with a minimum CT value are normally located on the outer side of the body boundaries of the patient's body 60. Their CT values are normally negative values. Because it is possible for various reasons that certain lines are not scanned in the course of the scanning operation of the topogram 200, or because a specific part of a certain line is not scanned, the CT values of these unscanned areas are exceptionally small. These are not suitable for use in the calculations corresponding to the last steps of the present invention. For this reason, a threshold value Tₘᵢₙ can be set for carrying out a comparison. The relevant Tₘᵢₙ value is set such that it is higher than the CT values of the points of the unscanned areas designated above, but lower than the CT values of the areas that have already been scanned without solid bodies. The minimum CT value found is compared with the Tₘᵢₙ value designated above. When the minimum CT value found is smaller than the relevant Tₘᵢₙ value, the latter is not considered as the minimum CT value of the relevant line. It is possible in this way to exclude the possibility of points from areas of the topogram 200 that have not been scanned for unexplained reasons from being considered as points with a minimum CT value.
d) Searching from points, described in step b), on the body bones in the vicinity of the body boundaries to the left—that is to say in a direction outside the body 60—for points whose CT value is the X2-fold of the minimum CT value designated above, 0.5<X2<1.
   It is known from numerous experiments that points whose CT value is the 0.5-1-fold of the minimum CT value designated above are located on the body boundaries of the patient's body 60. An optimum recognition effect is achieved in practical application when 0.25 is selected for the value X2. The reason why the search from the points of step b) to the body bones in the vicinity of the body boundaries is to be carried out to outside the patient's body 60 resides in that it is to be ensured in this way that it is possible to find the points which are located on the body boundaries of the body 60 and fulfill the CT value conditions designated above, and that the points which are located in the interior of the patient's body 60 and which fulfill the CT value conditions designated above are not found.
e) Repeating steps a) to d) to search for points on the body boundary inside the scan area designated above. These points form the body boundaries 62 of the patient's body 60.

The points for the right-hand body boundaries of the patient's body 60 of each scan line of the topogram can be found in a corresponding way with the aid of the same method. Consequently, the left-hand and right-hand body boundaries of the patient's body 60 are found and are used for the subsequent setting and reconstruction of the field of view.

Furthermore, the situation is such that when the maximum CT value designated above is greater in the step a) designated above than the predetermined metal CT value Tₘₑₜₐₗ, it is assumed that metallic material is located in the relevant scan line. Under these circumstances, the boundary points of the previous scan line are considered as boundary points of the relevant scan line. Because the boundaries of the human body normally have no sudden changes, the approximate solution designated above proves to be sensible and acceptable.

Furthermore, the situation is such that when no scanning operation has taken place for some lines in the topogram 200 designated above—as is the case in the area designated by the number 212—there is no possibility of finding the boundary point of the relevant scan line. Under these circumstances, it is possible to lengthen the boundary point of the last line just scanned, and to consider it as boundary 64 of the relevant scan line. It is possible in this way for the boundary 62 of the relevant patient's body 60 to be extended continuously and straightforwardly, and this is favorable for the further setting of the FOV.

With regard to lateral topograms, the search for the two boundaries takes place in accordance with the method designated above. This respectively involves the front boundary of the patent's body and the sickbed boundary. In fact, what is required by contrast is the front boundary of the patient's body and the rear boundary of the latter. The scanning operation is carried out under these circumstances with the patient lying flat on the sickbed. The rear boundary of the patient's body forms a straight line with an approximately parallel course in relation to the sickbed boundary. The spacing between the x-radiation source and sickbed is fixed, as is the thickness of the sickbed. For this reason, all that is required in order to obtain the rear boundary of the patient's body is to displace the sickbed boundary obtained in the direction of the patient's body in a horizontal fashion by a specific distance.

After the left-hand and the right-hand boundaries of the patient's body have been obtained or the front and rear sides of the patient's body have been obtained, the median filtering can optionally be performed in order to remove discrete points which have been found in the course of the search operation and correspond to the CT value conditions described under step c) and d).

Finally, the FOV is set on the left-hand and right-hand boundaries inside the scan area or on the front and rear boundaries such that the FOV contains precisely all boundary points inside the scan area designated above. Thus, the FOV area intersects the points of the outermost side of the boundary 62 of the patient's body 60 inside the scan area. In order to achieve an optimum effect of image reconstruction, the procedure as in FIG. 2 number 20′ is adopted. In the course of the setting of the FOV designated above, the positioning is set in a dynamic way while regulating the scan area, and while this is being done the FOV area intersects the points of the outermost side of the boundary 62 of the patient's body 60 inside the scan area.

After the setting of the scan area on the topogram 200 designated above and after setting of the FOV 20′, the serial or spiral scanning can subsequently be carried out.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for automatically setting and reconstructing a field of view along body boundaries on a CT topogram for setting and reconstructing the field of view inside a specific scan range on the topogram, the method comprising:
a) searching for a maximum CT value of each scan line inside a scan area on the topogram;
b) searching along the relevant scan line from outside the body in the direction inward for points whose CT value is the X1-fold of the maximum CT value, 0<X1<0.5, and the points being located on the body bones in the vicinity of the body boundaries;
c) searching from the points, described in step b), on the body bones in the vicinity of the body boundaries in the direction outside the body for a minimum CT value of the relevant scan line on the topogram;
d) searching from the points, described in step b), on the body bones in the vicinity of the body boundaries in the direction outside the body for points whose CT value is the X2-fold of the minimum CT value, 0.5<X2<1, and the points of the relevant scan line being located on the body boundaries;
e) repeating steps a) to d) to search for points on the body boundary inside the scan area; and
f) setting and reconstructing the field of view such that the field of view includes all found points on the body boundaries inside the relevant scan area.

2. The method for automatically setting and reconstructing the field of view along the body boundaries on a CT topogram as claimed in claim 1, wherein the points, described under step c), with the minimum CT value are located on the outside of the body boundary.

3. The method for automatically setting and reconstructing the field of view along the body boundaries on a CT topogram as claimed in claim 1, wherein, when the maximum CT value described under step a) is greater than a previously determined metal CT value, it is assumed that metal material is located in the relevant scan line, and wherein, in this case, the boundary points of the previous scan line are considered as boundary points of the present scan line.

4. The method for automatically setting and reconstructing the field of view along the body boundaries on a CT topogram as claimed in claim 1, wherein the data of a few lines of the start region and end region of the scan area of the topogram are erased in order to prevent deformation of linear artifacts.

5. The method for automatically setting and reconstructing the field of view along the body boundaries on a CT topogram as claimed in claim 1, wherein median filtering is further undertaken in order in the course of the search operation to remove found discrete points that correspond to the CT value conditions described under steps c) and d).

6. The method for automatically setting and reconstructing the field of view along the body boundaries on a CT topogram as claimed in claim 1, wherein, when setting and reconstructing the field of view in step f), the boundaries of the reconstructed field of view intersect the points of the outermost side on the body boundaries inside the scan area.

7. The method for automatically setting and reconstructing the field of view along the body boundaries on a CT topogram as claimed in claim 1, wherein X1 under step b) is selected as 0.25.

8. The method for automatically setting and reconstructing the field of view along the body boundaries on a CT topogram as claimed in claim 6, wherein X1 under step b) is selected as 0.25.

9. The method for automatically setting and reconstructing the field of view along the body boundaries on a CT topogram as claimed in claim 1, wherein X2 under step d) is selected as 0.75.

10. The method for automatically setting and reconstructing the field of view along the body boundaries on a CT topogram as claimed in claim 6, wherein X2 under step d) is selected as 0.75.

11. The method for automatically setting and reconstructing the field of view along the body boundaries on a CT topogram as claimed in claim 2, wherein median filtering is further undertaken in order in the course of the search operation to remove found discrete points that correspond to the CT value conditions described under steps c) and d).

12. The method for automatically setting and reconstructing the field of view along the body boundaries on a CT topogram as claimed in claim 2, wherein X1 under step b) is selected as 0.25.

13. The method for automatically setting and reconstructing the field of view along the body boundaries on a CT topogram as claimed in claim 11, wherein X1 under step b) is selected as 0.25.

14. The method for automatically setting and reconstructing the field of view along the body boundaries on a CT topogram as claimed in claim 2, wherein X2 under step d) is selected as 0.75.

15. The method for automatically setting and reconstructing the field of view along the body boundaries on a CT topogram as claimed in claim 11, wherein X2 under step d) is selected as 0.75.

16. A computer program to, when executed on a computer, cause the computer to carry out the method as claimed in claim 1.

17. A computer program product, including the computer program of claim 16.

18. A computer readable medium including program segments for, when executed on a computer, causing the computer to implement the method of claim 1.
